(19) United States
(12) Patent Application Publication
(10) Pub. No.: US 2010/0092419 A1
(43) Pub. Date: Apr. 15, 2010

(54) MAGNETIC FLUIDS AND THEIR USE

(76) Inventors: Carlos Guerrero-Sanchez, Eindhoven (NL); Mircea Rasa, Eindhoven (NL); Ulrich S. Schubert, Eindhoven (NL)

Correspondence Address: FERRELLS, PLLC, P. O. BOX 312, CLIFTON, VA, 20124-1706, US

(21) Appl. No.: 12/312,344

(22) PCT Filed: Oct. 6, 2007
(86) PCT No.: PCT/EP2007/009588

§ 371 Date: Oct. 28, 2009

(30) Foreign Application Priority Data
Nov. 7, 2006 (EP) PCT/EP2006/010654

(51) Int. Cl. H01F 1/26; H01F 1/00; A61K 31/74; A61K 31/715; A61K 31/717; A61K 47/30; A61K 47/26

(52) U.S. Cl. 424/78.08; 252/62.53; 252/62.54; 514/54; 514/57; 514/772.3; 514/777; 514/781

(57) ABSTRACT

Disclosed are magnetic fluids containing ionic liquids and containing no stabilisation agents or containing selected stabilisation agents. These magnetic fluids can be used in different fields of industry, for example as an ink, as a damping fluid, as a sealing fluid, in imaging applications, in sink flotation techniques, in biomedical applications, as a reaction medium to perform chemical reactions, as a reversible seal for occluding blood vessels in living organisms in medical therapy or as a transportation means and/or delivery means for chemical substances at a selected location within a chemical or biological system.

This invention relates to magnetic fluids containing ionic liquids. These magnetic fluids can be used in different fields of industry.

### BACKGROUND OF THE INVENTION

Magnetic fluids are suspensions or dispersions of magnetic particles in carrier fluids. The rheological properties of these magnetic fluids can be controlled by moderated magnetic fields. From this point of view magnetic fluids can be classified into ferrofluids and magnetorheological fluids (MRFs). Ferrofluids are stable colloidal dispersions of single-domain ferromagnetic or ferrimagnetic nanoparticles in a carrier fluid. The stabilization of the dispersion is based on steric repulsion provided by a surfactant of long chained molecules. As a consequence of their magnetic single-domains, ferrofluids only exhibit modest rheological changes under the influence of a magnetic field.

On the other hand MRFs are dispersions of small, for example micron or sub-micron sized magnetic particles in a carrier fluid. The main characteristic of MRFs is the manipulation of their rheological behavior by means of a magnetic field. Due to this property, MRFs can instantaneously and almost reversibly change from a liquid to a quasi-solid state.

MRFs have been proposed for many applications. They are proposed, for example, as semi-active shock absorbers for cars, as dampers for seismic damage controls for buildings and bridges, and as valves for robotic joint controls (compare I. Bica, J. Magn. Magn. Mater. 2002, 241, 196; and M. R. Jolly et al., Proc. SPIE—The International Society for Optical Engineering 1998, 262).

Moreover research in the medical field involving MRFs includes drug delivery and cancer therapeutic methods (compare U. O. Häfeli et al., J. Magn. Magn. Mater. 1999, 194, 76; J. Liu et al., J. Magn. Magn. Mater. 2001, 225, 209; and A. Meretei, Eur. Pat. Appl. EP 1676534).

Current fundamental research on MRFs focuses, mainly, on the settling problem of the dispersions (sedimentation and re-dispersion phenomena). In order to solve these problems several strategies have been proposed, for example addition of thixotropic agents, such as carbon fibers or silica nanoparticles; addition of surfactants, such as oleic or steric acid; addition of magnetic nanoparticles, the use of viscoplastic media as continuous phase, water-in-oil emulsions as carrier liquids, and polymeric core-shell structured magnetic particles.

J. D. Carlson, U.S. Pat. No. 6,132,633 discloses an aqueous magnetorheological material including besides water and magnetic-responsive particles bentonite or hectorite as anti-settling additives.

R. John et al., U.S. Pat. No. 6,875,368 discloses a magnetorheological fluid composition including castor oil as a carrier fluid, magnetic-sensitive particles coated with a selected particle stabilizer.

From the technological point of view there are several important aspects to be considered when designing magnetic fluids. In-use-thickening can be observed if an ordinary magnetic fluid is subjected to high stress and high shear rate over a long period of time. This means that an original low-viscosity magnetic fluid progressively shows a continuous increase in its viscosity until eventually it becomes an unmanageable paste having the consistency of shoe polish. Thus it would be desirable to provide a magnetic fluid that operates in the high shear regime of 10⁴ to 10⁶ sec⁻¹. Another important aspect to be considered when evaluating the quality of magnetic fluids are the conditions to which they will be exposed and not just the rheological behavior under normal laboratory conditions. Durability and life of magnetic fluids have found to be more significant barriers to commercial success than yield strength or the settling problem. In fact, except for very special cases such as seismic dampers, a complete stability against sedimentation is not a necessity. It is sufficient for most applications to have a magnetic fluid that soft settles i.e. a clear layer may form at the top of the fluid but the sediment remains soft an easily to be redispersed (e.g. magnetic fluid dampers and rotary brakes are efficient mixing devices and as long as the magnetic fluid does not settle into a hard solid, normal motion of the device is sufficient to re-disperse any sediment back to a homogeneous state). Moreover, for some applications, the attempt to make magnetic fluids absolutely stable against sedimentation may actually compromise their performance in a specific device.

So far, most of the magnetic fluids commercially available and the ones for research purposes have been prepared in limited carriers. These are, for example, mineral oil, hydrocarbon oils, synthetic oils including silicon oils, water, or glycols. These carriers are used in combination with a complex and expensive variety of proprietary additives, similar to those found in commercial lubricants to reduce gravitational settling and promote the suspension of the magnetic particles. On the one hand, these carriers may limit the potential applications of magnetic fluids in some specific areas of research and technology and, on the other hand, the use of expensive additives and methods of preparation to promote their stability increase their cost of production.

Ionic liquids (ILs) have appeared in recent years as novel compounds in materials research and are already used in several industrial processes. One of the main characteristics of ILs is the fact that their properties, such as viscosity, solubility, electric conductivity, melting point and biodegradation, can be tuned by varying the different involved anions and/or cations, which cannot be done for the conventional carriers of magnetic fluids. In addition, some other intrinsic properties related to the stability and “green” characteristics of ILs, such as negligible vapour pressure, negligible flammability and liquid state in a broad range of temperatures, turns them as very attractive materials to be investigated as carriers for magnetic fluids. Nowadays, more than 300 ILs are commercially available covering a wide range of physical and chemical properties.

Magnetic fluids containing ILs as carrier and containing no stabilizing additives or containing selected stabilizing additives as hereinafter defined have not been disclosed in the prior art.

JP-A-2006-193,686 discloses magnetic viscous fluids. These fluids contain magnetic particles which are stably dispersed for a long time in a liquid carrier by using inorganic whiskers as stabilization additives. Hence, JP-A-2006-193,686 discloses an invention of a magneto-viscous fluid characterized by containing magnetic particles stabilized with a basic magnesium sulfate whisker and/or a calcium silicate whisker in a dispersion medium. Among others, JP-A-2006-193,686 discloses the use of certain ionic liquids, represented by ethyl methyl imidazolium salt, 1-butyl-3-methyl imidazolium salt and 1-methyl-pirazorium salt, as possible dispersion media for the stabilization of magnetic particles with the basic magnesium sulfate whisker and/or the calcium silicate whisker.

In US-A-2004/003680 a decomposition method, in particular a complex chemical vapor deposition (CVD) method, for producing submicron metal containing particles in a liquid is disclosed. The metal containing particles can be produced direct in a liquid bath, e.g. for the preparation of MRFs. Preferred liquids are organic solvents. As another example for suitable liquids molten salts are mentioned. The use of ILs is not disclosed.

### SUMMARY OF THE INVENTION

It is an object of the present invention to provide a magnetic fluid which can be manufactured in an economic process using simple equipment and which results in stable dispersions against sedimentation of magnetic particles in a carrier liquid even without use of additives to stabilize the dispersion.

Surprisingly it has been found that when using ILs or mixtures thereof as carriers for magnetic fluids, dispersions of high stability against sedimentation are obtained and the addition of stabilisation agents can be avoided.

Even though the magnetic dispersions of this invention show good stability against sedimentation in the absence of stabilization agents, these dispersions may include some stabilization agents as an improvement to the sedimentation problem and aiming at the development of a completely stable dispersion against sedimentation.

The magnetic fluids obtained show a low sedimentation rate even in the absence of any stabilizing agent, and depend, mainly, on the type of IL utilized as a carrier.

Furthermore, the magnetic fluids obtained show a very low vapour pressure, negligible flammability, a liquid state and stability (chemical and physical) over a broad temperature range, electric conductivity, and their miscibiliy or immiscibility with other substances can be tuned.

### DETAILED DESCRIPTION OF THE INVENTION

The present invention relates to a magnetic fluid comprising magnetic particles in an ionic liquid or in a mixture of ionic liquids and which magnetic fluid does not contain stabilisation agents or which contains stabilisation agents selected from the group of carbon fibers, natural or synthetic water-soluble thixotropic agents, resins, starches, polysaccharides, cellulose derivatives, sodium tetraborate decahydrate, seaweed extracts, synthetic resins, surfactants, viscoplastic media, water-in-oil emulsions or a combination of two or more thereof.

The magnetic fluids of this invention encompass ferrofluids and MRFs, while the latter being preferred.

The term “ionic liquid” (IL) as used in this specification shall mean a composition of matter being liquid at temperatures below 200° C. and consisting essentially of cations and anions to form an electroneutral composition of matter. In general at least 85% by weight, preferably at least 95% by weight of an IL consists of cations and anions the remainder may consist of electrically neutral species. An IL for use in the fluids of this invention in general is a composition of matter having a melting temperature between −100° C. and 200° C. ILs are salts or their mixtures consisting of at least one organic component, ILs can be miscible or immiscible with water and reactive or non-reactive with water, air or other chemical species depending on their structure. The selection of a proper IL will depend on the desired physical and chemical properties of the prepared magnetorheological fluid (MRF). Variation in cations and anions can produce a very wide range of ILs allowing for the fine-tuning of their physical and chemical properties for specific applications. The control on characteristics, such as hydrophilicity, can be obtained by changing the anion and a fine control on their properties can be obtained by selecting a proper alkyl group on the cation. The constituents of ILs are constrained by high columbic forces and therefore exert negligible vapor pressure above the liquid surface. ILs show also negligible flammability under certain conditions (see for example M. Deetlefs et al., Chim. Oggi 2006, 24(2), 16).

A wide variety of ILs suitable for use in the fluids of the present invention have been described. Examples of ILs suitable for the fluids of the present invention are given in V. R. Koch et al., U.S. Pat. No. 5,827,602; F. G. Sherif et al., U.S. Pat. No. 5,731,101; H. Olivier et al., U.S. Pat. No. 5,892,124; and in T. Welton, Chem. Rev. 1999, 99, 2071).

The term IL as used in this specification can also include a class of less expensive ILs that may be used as co-solvents to lower the overall cost of the carrier systems of the fluids, therefore mixtures of ILs are contemplated and within the scope of the present invention. For example, N,N′-dialkylimidazolium bistrifylimide salts are expensive, but they can be mixed with tetraalkylammonium-based salts to obtain less expensive ILs. The mixtures are characterized to establish how this affects the physical and chemical properties of the IL mixtures.

Preferred magnetic fluids contain an IL consisting to at least 95% by weight of ions and being liquid at temperatures between −100 and +200° C.

Cations of ILs are typically large, bulky, and asymmetric, and show a direct influence on the melting point of the IL. Common examples of cations include organo-ammonium, organophosphonium and organosulfonium ions, such as N-alkylpyridinium, N-alkyl-vinylpyridinium, tetraalkylammonium, tetraalkylphosphonium, trialkylsulfonium, N,N′-dialkylimidazolium, N-alkyl(aralkyl)-N'-alkylimidazolium, N-alkyl-N'-vinylimidazolium, pyrrolidinium and the AMMOENG™ cation series from Solvent-Innovation GmbH. Other heterocycles containing at least one quaternary nitrogen or phosphorus or at least one tertiary sulfur are also suitable. Examples of heterocycles that contain a quaternary nitrogen include pyridazinium, pyrimidinium, oxazolium and triazolium ions.

Particularly, preferred cations are N-alkylpyridinium, tetraalkylammonium, tetraalkylphosphonium, trialkylsulfonium, and N,N′ dialkylimidazolium, because of their low cost, ease of preparation, ready availability, and stability.

By varying the number of carbons and branching on the alkyl chains of these cations, the melting temperature of the IL and therefore the melting temperature of the corresponding MRF and other physical properties, such as viscosity, can be easily be adjusted to a desirable value (see for example P. Bonhote et al., Inorg. Chem. 1996, 35, 1168; S. V. Dzyuba et al., Chemphyschem, 2002, 3, 161; G. Law et al., Langmuir, 2001, 17, 6138-6141).

Preferably, the cations have alkyl chains from 2 to 20 carbon atoms, more preferably from 4 to 10 carbon atoms.

Cations can also be double charged species such as those described in the literature (see for example M. J. Muldoon et al., J. Polym. Sci. Pol. Chem. 2004, 42, 3865). Cations can also react with other chemicals species, i.e. they can react with each other to form polymers as described in literature (see for example R. Marcilla et al., Macromol. Chem. Phys. 2005, 206, 299; M. J. Muldoon et al., J. Polym. Sci. Polym. Chem. 2004, 42, 3865) in order to modify the prepared MRFs based on ILs.

Preferably at least one cation forming said IL is an ammonium cation with at least one organic group or a phosphonium cation with at least one organic group or a sulfonium with at least one organic group or a quaternary nitrogen atom containing heterocyclic group, for example a pyridinium salt or an imidazolium salt.

The anions present in these liquids can be selected of either anion.

Anions of ILs can be complex inorganic anions that are non coordinating with respect to the organic cation and non-interfering with respect to the cationically active species. Many suitable anions are conjugated bases derived from protic acids having a pKₐ of less than 4, for example tetrafluoroborate, the conjugate base of fluoroboric acid, which has a pKₐ<5).

Other suitable anions are adducts of a Lewis acid, trihalides and a halide, such as tetrachloroaluminate, or single halides, such as fluoride, chloride, bromide and iodide. Suitable anions include also, for example, hexafluorophosphate, hexafluoroantimonate, hexafluoroarsenate, tetrafluoroborate, dicyandiamide, methanesulfonate, tosylate tetrachloroborate, tetraarylborates, polyfluorinated tetraarylborates, tetrahalo-aluminates, alkyltrihaloaluminates, triflate (CF₃SO₃⁻), nonaflate (CF₃(CF₂)₃SO₃⁻), bistrifylimides (bis(trifluoromethylsulfonyl)imides), (bis(perfluoroethylsulfonyl)imides), (bis(trifluoroethylsulfonyl)methides), chloroacetate, trifluoroacetate, alkylsulfates, N—(N-methoxyethoxy)alkylsulfates, dialkylphosphates, [MeCO₂]⁻, nitrate, nitrite, trichlorozincate, dichlorocuprate, fluorosulfonate, triarylphosphine, sulfonates, and polyhedral boranes, carboranes, and metallocarboranes.

The anion contributes to the overall characteristics of the IL including physical and chemical properties, such as chemical stability in presence of air and water and solubility with other substances (see for example A. Bagno et al., Org. Biomol. Chem. 2005, 3, 1624; P. Bonhote et al., Inorg. Chem. 1996, 35, 1168; R. Marcilla et al., Macromol. Chem. Phys. 2005, 206, 299). For example, in general, ILs containing chloride anions are hydrophilic and ILs containing hexafluorophosphate anions are hydrophobic. An advantage of the bistrifylimide anion is that these provide ILs with viscosities and densities similar to water making them easy to work with.

A preferred magnetic fluid contains an imidazolinium salt and/or a phosphonium salt as an IL, very preferred a 1-alkyl-3-alkylimidazolinium salt and/or a tetraalkyl-phosphonium salt and/or a tetraalkylammonium salt.

The amount of the IL or mixture of ILs in the fluids of this invention is in the range between 40 and 99% by weight, preferably between 60 and 95% by weight, referring to the total composition of the fluid.

The term “magnetic particle” as used in this specification shall mean a composition of matter in particulate form which shows ferromagnetic, ferrimagnetic, antiferromagnetic, canted-spin ferromagnetic, paramagnetic and superparamagnetic properties.

Ferromagnetic materials contain magnetic domains in each of which the magnetic moments of individual atoms are oriented in the same direction. When the domains are randomly oriented the total magnetic moment of the ferromagnetic material is zero. When the moments have a preferred orientation, the total moment is non-zero and the substance is “magnetized”. The magnetic domains are separated by domains walls of finite size or crystal boundaries. Such a domain wall represents an interfacial free energy cost, which competes with the (bulk) domain formation which is favourable because it lowers the internal magnetic energy. At large material volumes the bulk term dominates and a multi-domain structure is formed. However, below a critical particle volume formation of domain walls no longer occurs. The particle is then a so-called mono-domain particle. When the direction of the moment is thermally oscillating, these mono-domain particles are the magnetic analogues of paramagnetic ions. However, their magnetic moment is much larger than for “ordinary” paramagnetic materials. Therefore this phenomenon is called superparamagnetism. In an external magnetic field the total magnetic moment of a ferromagnetic material may increase. This is caused by two effects. Firstly, by the growth of domains with magnetic moments in the same direction as the magnetic field, at the expense of their neighbors. The second effect is that magnetic dipoles in other domains will rotate towards the direction of the field. The magnetization behavior of a ferromagnetic material with an initial magnetic moment of zero to its saturation magnetic moment, Mₛ, is given in FIG. 3 described below. Hysteresis is observed upon reduction of the magnetic field due to slow rearrangement of the domains (compare FIG. 3). A zero magnetic field a residual magnetisation, Mᵣ, remains (compare FIG. 3). The coercive field, H_{c}, is the field where the total magnetic moment becomes zero again (compare FIG. 3). The ferromagnetic order tends to be disrupted by thermal agitation. The Curie temperature, T_{c}, is the temperature above which the disruption is complete so that the domains loose their magnetization. Ferromagnetism is exhibited by iron, nickel, cobalt and many of their alloys. Some rare earth elements, such as gadolinium and certain intermetallics, like gold-vanadium, are also ferromagnetic substances.

In antiferromagnetic materials the spins of one set of atoms are antiparallel aligned to another set. When these magnetic moments are equal the net magnetic moment is zero. The antiferromagnetic order disappears at the Néel temperature. Antiferromagnetism is a property of MnO, FeO, NiO, FeCl₂ and many other compounds (see for example R. E. Rosenweig, Ferrohydrodynamics, Cambridge University Press, Cambridge, 1985).

Canted-spin ferromagnetism is a weak or parasitic form or ferromagnetism. Due to a small deviation from antiferromagnetic order (canting of moments) a small magnetic moment is generated. A well-known material exhibiting this type of magnetism is hematite (α-Fe₂O₃).

Ferrimagnetism is caused by the presence of two or more different types of lattice sites (for example octahedral and tetrahedral as in spinel) which are occupied by ions with different magnetic moments. These magnetic moments are aligned antiparaliel and because of the difference in magnetic moment a net magnetic moment results. Thus a ferrimagnet is externally almost identical to a ferromagnet and can be said to exhibit ferromagnetic behavior. But microscopically the order more closely resembles antiferromagnetism since neighboring moments are antiparaliel. Examples of ferrimagnetic materials are ferrites with the general formula MO.Fe₂O₃, where M stands for Fe, Ni, Mn, Cu or Mg (see for example R. E. Rosenweig, Ferrohydrodynamics, Cambridge University Press, Cambridge, 1985). Usually the term ferromagnet also comprises materials which are actually ferrimagnetic. A familiar example is magnetite (Fe₃O₄)

Atoms which contain unpaired electrons, such as liquid oxygen or rare-earth salt solutions and ferromagnets above the Curie temperature will show paramagnetic behavior. In zero magnetic field the dipoles are randomly oriented. However in a magnetic field the torque on the dipoles tends to align them with the field. This alignment usually is not complete because of disruptions by thermal motions. The magnetization depends linearly on the applied magnetic field and reduces to zero on removal of the field.

The terms related to the stability of the MRFs of this invention, used throughout this document, refer to the stability of the MRFs against gravitational sedimentation of the dispersed magnetic particles in the respective ILs, unless another definition of stability is given.

Magnetic particles used in the fluids of this invention can have particle sizes that result in especially stabilized fluids. For example, the use of extremely bimodal iron-magnetic particles have been used for the preparation of stable MRFs (M. T, Lopez-Lopez et al., J. Mater. Res. 2005, 20, 874). Other examples of the use of magnetic nanoparticles for stable MRFs are given in literature (see for example B. D. Chin et al., Rheol. Acta, 2001, 40, 211).

Magnetic particles used in the fluids of this invention can be combined with polymers to result in especially stabilized fluids. For example, core-shell structured magnetic particles with different polymers, such as with poly(methylmethacrylate) or with polystyrene, can be fabricated by a dispersion polymerization method. These core-shell structured magnetic particles have been already used to enhance the dispersion stability of conventional MRFs when dispersed in mineral oil (J. S. Choi et al., J. Magn. Magn. Mater. 2006, 304, e374).

The magnetic particles can have different shape, for example regular shapes, such as sphere, disc, platelet, fibre, or irregular shapes. Mixtures of different magnetic particles can be used.

Preferred magnetic particles have average diameters of below 100 μm, very preferred in the range between 10 nm and 50 μm, and most preferred between 100 nm and 20 μm. The average diameters of the particles can be obtained by standard image analysis techniques as reported in the literature (see for example C. Guerrero-Sanchez et al., Chem. Eur. J. 2006 DOI: 10.1002/chem. 200600657).

Preferred magnetic particles are ferromagnetic or ferrimagnetic and can consist of either ferromagnetic and/or ferrimagnetic materials.

Preferred examples of magnetic particles include iron, carbonyl iron, iron alloy, iron oxide, iron nitride, iron carbide, low-carbon steel, nickel, cobalt, rare-earths, such as gadolinium or mixtures thereof or alloys thereof.

Very preferred magnetic particles consist of iron, iron oxides, cobalt, nickel, gadolinium and their ferromagnetic or ferrimagnetic alloys.

The amount of the magnetic particles in the magnetic fluids of this invention can vary over a broad range. Typically these magnetic particles are present in the fluid in an amount between 1 and 60% by weight, referring to the total amount of the magnetic fluid. Preferred amounts of magnetic particles in the magnetic fluids are between 5 and 40% by weight.

The magnetic fluids of this invention can be prepared by simply mixing the ingredients in a mixing equipment known in the art.

Equipment known in the art can include mechanical stirring, rotatory drums or ultra-sonication techniques. Preferably devices made of non-magnetic material, such as polyethylene, polypropylene or other polymers, can be used during the mixing process to avoid interactions of the magnetic particles with the mixing equipment.

A more preferable method is provided by mechanical stirring with stirring rates above 100 rpm and a stirring time of at least 1 second or the time necessary to obtain a homogeneous dispersion at certain stirring rate.

The temperature of the mixing process can vary over a broad range. The mixing temperature can vary from super-cooled conditions (some degrees below the freezing temperature of the used IL or mixture of ILs) up to the decomposition temperature of the used IL or mixture of ILs. If magnetic particles are mixed the temperature must be below the Curie or Néel temperature of the utilized magnetic particles. The mixing process can be performed under a wide range of pressures (from high vacuum conditions up to high pressures, including atmospheric pressure) due to the fact that most of ionic liquids show negligible vapour pressure.

While the use of stabilisation agents (=additives) is not preferred the magnetic fluids of this invention may contain such additives. Examples thereof are selected thixotropic agents, surfactants, viscoplastic media, water-in-oil emulsions or a combination of two or more thereof.

The amount of additives in the fluids of this invention is in the range between 0.1 and 40% by weight, preferably between 1 and 15% by weight, referring to the amount of IL or mixture of ILs.

Examples of thixotropic agents are carbon fibers. These have been added to a conventional MRF to improve its stability against settling (S. T. Lim et al., J. Magn. Magn. Mater. 2004, 282, 170); the mentioned additive is commonly used as thickening, antisag, thixotropy and anti-settlement of heavy pigments in paint industry.

Additional examples of thixotropic agents are natural or synthetic water-soluble thixotropic agents, such as gums (e.g. arabic, ghatti, karaya, tragacanth, guar, locust bean, quince seed, psyllium seed and flax seed), resins, starches, polysaccharides, cellulose derivatives, sodium tetraborate decahydrate or mixtures of any of the foregoing, seaweed extracts (e.g. agar, algin, carrageenan, fucoidan, furcellaran, laminarin, hypnean, porphyran, funoran, dulsan, iridophycan or hydrocolloids), and synthetic resins (e.g. polyethylene imines, polyacrylamide, polyvinyl alcohol, pyrrolidone based polymers and acrylic resins), which have been used for the stabilization of MRFs (J. D. Carlson, U.S. Pat. No. 6,475,404).

Examples of surfactants are long-chain alkanoic acids or alkenoic acids, such as stearic acid and other polymeric surfactants. For example, grafted maghemite with fatty acids have been used for the preparation of magnetic dispersions (G, A. van Ewijk et al., J. Magn. Magn. Mater. 1999, 201, 31). The use of polyethers for dispersing magnetic particles has been described also in the literature (K. Hata et al., U.S. Pat. No. 6,780,343).

The use of siloxanes terminated with hydroxyl or alkoxy groups which react directly with the magnetic particles has been also investigated to stabilize magnetic dispersions (P. P. Phule, U.S. Pat. No. 6,712,990).

Magnetically responsive foams in different liquids carriers containing different polymeric foams (polyurethanes) have been described in the literature (E. W. Purizhansky, U.S. Pat. No. 6,673,258).

Other examples of surfactant agents for MRFs are given in literature (see for example A. Dang et al., Ind. Eng. Chem. Res. 2000, 39, 2269; P. P. Phule et al., J. Mater. Res. 1999, 14, 3037; O. O. Park et al., U.S. Pat. No. 6,692,650; J. H. Park et al., J. Colloid Interface Sci. 2001, 240, 349).

Examples of viscoplastic media are greases that can be used—besides ILs or mixtures of ILs—as continuous phase of the MRFs of this invention. For example, the use of a commercial grease (e.g. Quaker State NLGI no. 2) and of a grease containing mineral oil and stearic acid as main components have been described as viscoplastic continuous media for the preparation of MRFs (P. J. Rankin et al., Rheol. Acta, 1999, 38, 471).

Water-in-oil emulsions as carrier liquids of MRFs have been already proposed in the literature (J. H. Park et al., J. Colloid Interface Sci. 2001, 240, 349; O. O. Park et al., U.S. Pat. No. 6,692,650). These water-in-oil emulsions can be used—besides ILs or mixtures of ILs—as continuous phase of the MRFs of this invention

The magnetic fluids of this invention can be used in different fields of industry.

Non-limiting examples are the use of these fluids as an ink, preferably for ink-jet printing; the use as a damping fluid, preferably for loudspeakers, graphic plotters or instrument gauges; the use as a sealing fluid, preferably for gas lasers, motors, blowers or hard drivers; the use in imaging applications, preferably for domain observations or as contrast agents; the use in sink flotation techniques, preferably in the recovery of resources from waste materials; the use in biomedical applications, preferably for drug targeting, cell labeling or attached drugs tomagnetic particles; the use as a reaction medium to perform chemical reactions, for example to control the diffusion of the involved reactants by the means of controlling the viscosity of the reaction media with a magnetic field; the use in the formation of reversible seals for occluding blood vessels in living organisms in medical therapy; or the use of the fluids of this invention containing additional chemicals substances, such as reactants or catalysts, for transportation and/or delivery of said chemical substance at a selected location within a chemical or biological system by means of a controlled magnetic field, e.g. within multi-phase, interfacial, biological, homogeneous or heterogeneous reaction system.

These uses are also object of the present invention.

An example for the transportation and/or delivery of chemical substances in chemical systems consists of a bi-phase system containing an upper phase of cyclohexane and a lower phase of water. Subsequently, a drop of a fluid of this invention containing a specific amount of calcium hydride (CaH₂), which reacts with water to release hydrogen (H₂), is introduced into the reaction system. In the case of a MRF based on a hydrophilic IL, the magnetic dispersion passes intact through upper phase (due to the fact that the used IL does not mix with cyclohexane) to reach the bottom phase (water), where the IL in the MRF starts to dissolve in the aqueous phase and therefore the original magnetic dispersion is destroyed at this point. Therefore CaH₂ is released rapidly and reacts with water to form H₂. The release of CaH₂ can be accelerated by approaching and moving a magnetic field next to the reaction system.

In another example for the transportation and/or delivery of chemical substances, in a chemical reaction system containing an upper phase of cyclohexane and a lower phase of water, a MRF based on a hydrophobic IL was used. Similar to in the previous example, here the magnetic dispersion again passed intact through the upper phase (due to the fact that the used IL did not mix with cyclohexane) to reach the bottom phase (water), in this case the MRF did not dissolve into the water but CaH₂ was still released slowly and reacted with the surrounding water to form H₂. In this system the position of the releasing of CaH₂ in the reaction system could be controlled by approaching and moving a magnetic field to a selected position.

An example for the use of the MRFs of this invention as reaction media consists of performing polymerization reactions using the described MRFs of this invention as a reaction medium. This can be performed using a similar approach to that one described in the literature (C. Guerrero-Sanchez et al., Chem. Commun., 2006, 3797) for polymerization reactions performed in ILs as reaction media. For the purposes of the applications of the MRFs of this invention, chemical reactions can be performed using different MRFs based on ILs as reaction media, thereafter the obtained products can be isolated from the reaction medium and the corresponding MRFs can be recovered in order to perform further reaction cycles using a suitable separation process. In another example, the MRFs of this invention can be used as reaction media to perform polymerization reactions in order to synthesize MRFs-polymer composites consisting of the dispersed magnetic particles in the corresponding IL or mixture of ILs mixed with a polymer matrix. The resulting polymer composites have shown magnetic properties and are electrical conductors. The polymer composites are resistant to fire owing to the presence of ILs in the polymer matrix, which have the properties of flame retardants due to the fact ILs have negligible flammability. In another method for the preparation of the described MRFs-polymer composites, the MRFs described in this invention can be mixed directly with polymers or oligomers synthesized in another step using methods known in the art such as extrusion, reactive extrusion, injection molding and solution.

In addition the invention relates to the use of a IL or a mixture of ILs for the preparation of magnetic fluids.

### EXAMPLES

In the following examples the following materials were used:

Magnetic particles I: Iron(II, III) oxide (magnetite) powder (<5 μm, 98%, density 4.8-5.1 g cm⁻³ (25° C.), Aldrich)
Magnetic particles II: Magnetite nano-powder (spherical, 20-30 nm, >98%, density 0.84 g cm⁻³, Aldrich)
Ionic liquid 1: 1-ethyl-3-methylimidazolium diethylphosphate
Ionic liquid 2: 1-butyl-3-methylimidazolium hexafluorophosphate
Ionic liquid 3: 1-hexyl-3-methylimidazolium chloride
Ionic liquid 4: 1-butyl-3-methylimidazolium trifluoromethanesulfonate
Ionic liquid 5: 1-butyl-3-methylimidazolium tetrafluoroborate
Ionic liquid 6: AMMOENG™ 100
Ionic liquid 7: 1-ethyl-3-methylimidazolium ethylsulfate
Ionic liquid 8: trihexyltetradecylphosphonium chloride

All ILs were synthesis grade and dried under vacuum at 40° C. at least 1 day before using them. Table 1 summarizes some properties of the ILs used.

TABLE 1

| | density | melting | viscosity | conductivity | |
|---|---|---|---|---|---|
| Ionic liquid | (g cm⁻³) | point (° C.) | (mPa s) | (mS cm⁻¹) | water soluble |
| 1-Ethyl-3- | 1.14 | — | — | — | yes |
| methylimidazolium | | | | | |
| diethylphosphate (1) | | | | | |
| 1-Butyl-3- | 1.36 | 11 | 196 | 1.34 | no |
| methylimidazolium | | | | | |
| hexafluorophosphate | | | | | |
| (2) | | | | | |
| 1-Hexyl-3- | 1.05 | −75 | 7827 | 0.3 | yes |
| methylimidazolium | | | | | |
| chloride (3) | | | | | |
| 1-Butyl-3- | 1.3 | 17 | 113 | 0.37 | yes |
| methylimidazolium | | | | | |
| trifluoromethane- | | | | | |
| sulfonate (4) | | | | | |
| 1-Butyl-3- | 1.21 | −71 | 119 | 3.53 | yes |
| methylimidazolium | | | | | |
| tetrafluoroborate (5) | | | | | |
| AMMOENG ™ 100 | 1.10 | <−65 | 1665 | — | gel |
| (6) | | | | | formation |
| 1-Ethyl-3- | 1.24 | <−65 | 109 | 3.95 | yes |
| methylimidazolium- | | | | | |
| ethylsulfate (7) | | | | | |
| Trihexyltetradecyl- | 0.89 | −70 | 2454 | — | no |
| phosphonium- | | | | | |
| chloride (8) | | | | | |

### MRF Preparation Method

The preparation of the MRFs using ILs as carriers was performed by mixing the corresponding IL with the magnetite particles. The compositions of the different prepared MRFs are summarized in Table 2. The mixing process was performed in cylindrical polyethylene containers using polyethylene stirring paddles in order to avoid interactions with the suspended magnetic particles. The mixing process was performed by mechanical agitation using a stirring rate of 2400 rpm for 15 minutes at room temperature (21° C.).

TABLE 2

| | | % wt/ | | | |
|---|---|---|---|---|---|
| | Ionic | mag- | | saturation | remanence |
| MRF | Liquid | netic | density | magnetization | magnetization |
| No. | (carrier) | particle | (g cm⁻³) | (kA m⁻¹) | (kA m⁻¹) |
| MRF1 | 1 | 25/I | 1.40 | 27.9 | 8.95 |
| MRF2 | 1 | 25/II | — | — | — |
| MRF3 | 2 | 25/I | 1.67 | 34.4 | 9.40 |
| MRF4 | 3 | 25/I | 1.23 | 23.7 | 7.09 |
| MRF5 | 4 | 25/I | 1.58 | 33.7 | 9.6 |
| MRF6 | 5 | 25/I | 1.48 | 30.3 | 8.84 |
| MRF7 | 6 | 25/I | — | — | — |
| MRF8 | 7 | 25/I | 1.51 | 32.3 | 10.0 |
| MRF9 | 8 | 25/I | 1.12 | 24.1 | 7.93 |
| MRF10 | 8 | 8.5/I | 0.95 | 7.03 | 2.40 |
| MRF11 | 8 | 2/I | 0.91 | — | — |
| MRF12 | 8 | 0.2/I | 0.89 | 0.13 | 0.04 |

### Characterization Techniques

Sedimentation measurements were performed under gravitational field in a similar way as described in the literature. The same volume amount of the prepared MRFs were poured into cylindrical polyethylene tubes of 4 mm diameter and 53 mm length and closed. The tubes were placed on a heavy marble table to minimize vibrations. The experimental set-up was placed in a room with controlled temperature (21° C.). Before starting the measurements it was checked if the tubes were standing perfectly vertical. The dispersion-IL interface (e.g. supernatant clear layer formation) was monitored by bare eye.

Magnetization measurements were carried out using an alternating gradient magnetometer (MicroMag 2900) at room temperature (21° C.). The corresponding MRFs were placed in the sample holders of the equipment and weighted just before the measurements. The volumes of the measured samples were obtained from the weight of the samples and the densities of the corresponding MRFs. The densities of the different MRFs were measured with a picnometer at 21° C. (obtained experimental values are shown in Table 2).

Magnetorheological measurements of the prepared MRFs were performed at 25° C. and under steady shear (at different shear rates) using a Physica MCR500 rheometer (Anton Paar) coupled with the commercial magnetorheological device MRD180-C (magnetorheological cell PP20/MR). The coil current and magnetic field strength were controlled using a separate control unit and the rheometer software (US 200, Physica Anton Paar). The homogeneous magnetic field was oriented perpendicular to the shear flow direction. A parallel-plate measuring system with a diameter of 20 mm which was made of non-magnetic metal to prevent the occurrence of radial component of magnetic forces on the shaft of the measuring system was used.

Images of some of the MRFs were recorded with an optical light microscope using an axioplan imaging 2 (Zeiss). The samples were placed between glass microscope slides in order to perform the imaging.

Thermogravimetric (TGA) analyses of the investigated ILs were performed on a Netzsch TGA 209 F1 instrument using nitrogen as the purge gas. The utilized heating rate was 10° C./min and the analyses were performed over a temperature range of 30 to 900° C.

Before performing all the mentioned characterization methods, the prepared MRFs were additionally homogenized by vigouros shaking. After shaking the MRFs showed no inhomogeneities (e.g. no supernatant clear layer formation) for a considerable time as sedimentation measurements revealed.

### BRIEF DESCRIPTION OF THE FIGURES

FIG. 1 shows the results of the sedimentation measurements of some of the MRFs of this invention.

In FIG. 2 the influence of the concentration of the dispersed magnetic particles as well as their size on the stability of the prepared MRFs is shown.

FIG. 3 shows a representative magnetic hysteresis loop of the obtained magnetic measurements of one MRF of this invention.

In FIG. 4 the magnetic moments for an IL used in this invention and for a combination of IL and sample holder is shown.

In FIG. 5 it is demonstrated that for fluids in which the content of dispersed magnetic material is very low, even the weak magnetic properties shown by the sample holders and the carriers can have a considerable influence on the determination of the magnetic properties of the sample.

In FIG. 6 the viscosity and shear stresses of MRFs of this invention are illustrated.

FIG. 7 reveals that the rheological properties of the prepared MRFs can be modified by the means of a magnetic field.

In FIG. 8 the influence of the content of the dispersed magnetic particles in the investigated MRFs on the rheological properties in the absence and in the presence of a magnetic field is depicted.

In FIG. 9 it is demonstrated that the modification of rheological properties of the analyzed MRFs is a reversible process.

In FIG. 10 optical microscopy images for MRFs of the present invention are shown.

FIG. 11 displays results of thermogravimetric measurements for the ILs used for the preparation of the MRFs of this invention

### RESULTS AND DISCUSSION

Some of the measured properties of the magnetic fluids prepared are shown in Table 2 above.

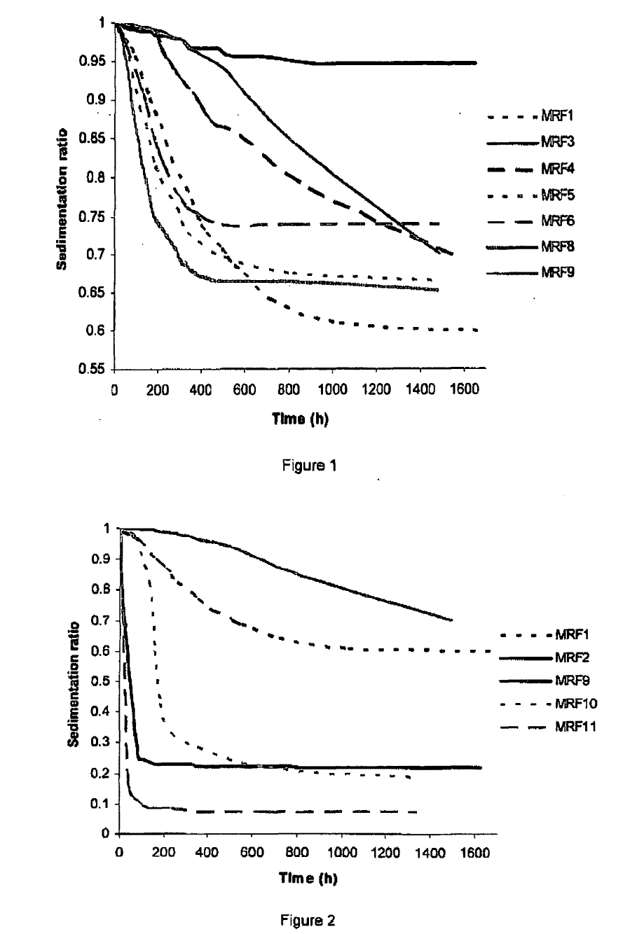

FIG. 1 shows the results of the sedimentation measurements of some of the MRFs of table 2 revealing, in general, a low sedimentation rate for most of the analyzed cases.

As shown in FIG. 1, carrier IL2=1-butyl-3-methylimidazolium hexafluoro-phosphate showed an outstanding stability against sedimentation of the dispersed magnetic particles (sedimentation ratio of 0.95 over a period of 70 days (1680 h)). Therefore this IL is very preferred for applications where the stability of the dispersion for long periods of time is an important factor to be considered (e.g. seismic dampers).

The influence of the concentration of the dispersed magnetic particles as well as their size on the stability of the prepared MRFs is shown in FIG. 2. For the case of the particle size, it was observed that the use of magnetic nano-particles during the preparation of the described MRFs leads to dispersions with a more rapid sedimentation rate (MRF2).

Surprisingly, however, when particles in the micrometer range were used, at the same % wt., the stability of the dispersion improved considerably (MRF1).

FIGS. 1 and 2 show that the use of ILs as carriers in dispersions of micron-sized magnetic particles allow for the preparation of MRFs with improved stability without utilizing any stabilizing additives. Regarding the concentration effect of the particles on the stability of the dispersions, FIG. 2 reveals that the content of particles shows an inverse relation to the rate of sedimentation. Thus, MRF9 (25% wt. of dispersed micron-sized magnetic particles in the corresponding IL) showed a considerable lower sedimentation rate than MRF11 (2% wt. of dispersed micron-sized magnetic particles in the corresponding IL); an intermediate case, MRF10 (8.5% wt. of dispersed micron-sized magnetic particles in the corresponding IL), is also shown in FIG. 2. With regard to MRF2 and MRF7, these samples were not completely investigated owing to their lower stability characteristics. MRF2 revealed poor stability against sedimentation as shown in FIG. 2. MRF7 showed a considerable “In-Use-Thickening” during the described preparation method, becoming an unmanageable paste having the consistency of shoe polish. However, this latter sample recovered its original consistency after a period of days at rest and, therefore some rheological measurements could be performed as discussed later on.

Table 2 summarizes the measured densities of the prepared MRFs as well as some of their magnetic characteristics (saturation magnetization (Mₛ) and remanent magnetization (Mᵣ)) as obtained from the magnetization measurements. The coercive field (Hₑ) of all measured MRFs was around −13 kA m⁻¹ except for MRF12 (sample with the less content of magnetite) which revealed a value of −8.9 kA m⁻¹.

The magnetic characteristics of the MRFs reported in Table 2 were calculated dividing the magnetic moments of the samples (as obtained for the magnetic measurements) by the corresponding volumes of the samples (which were estimated from the weights of the analyzed MRFs and their respective densities).

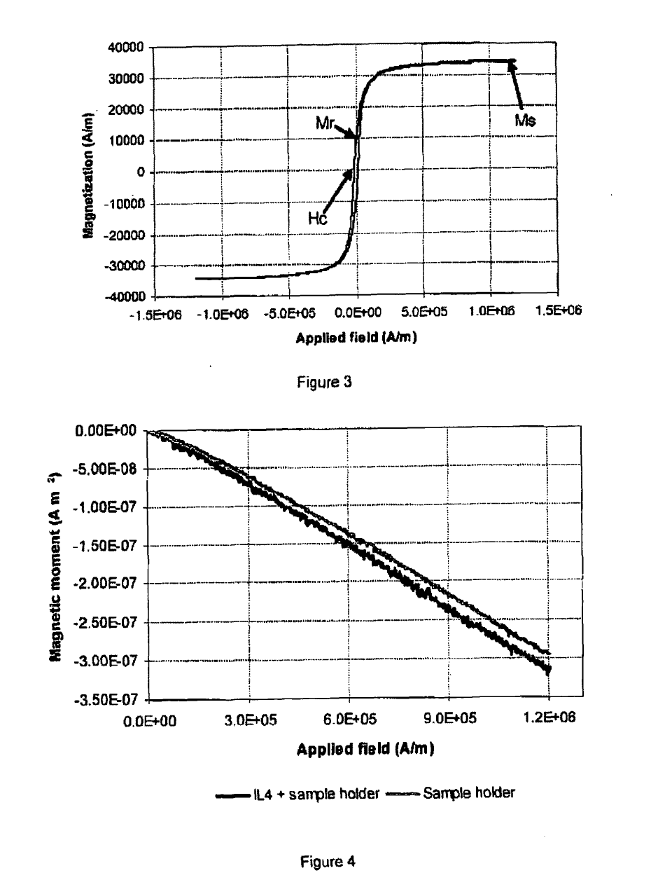

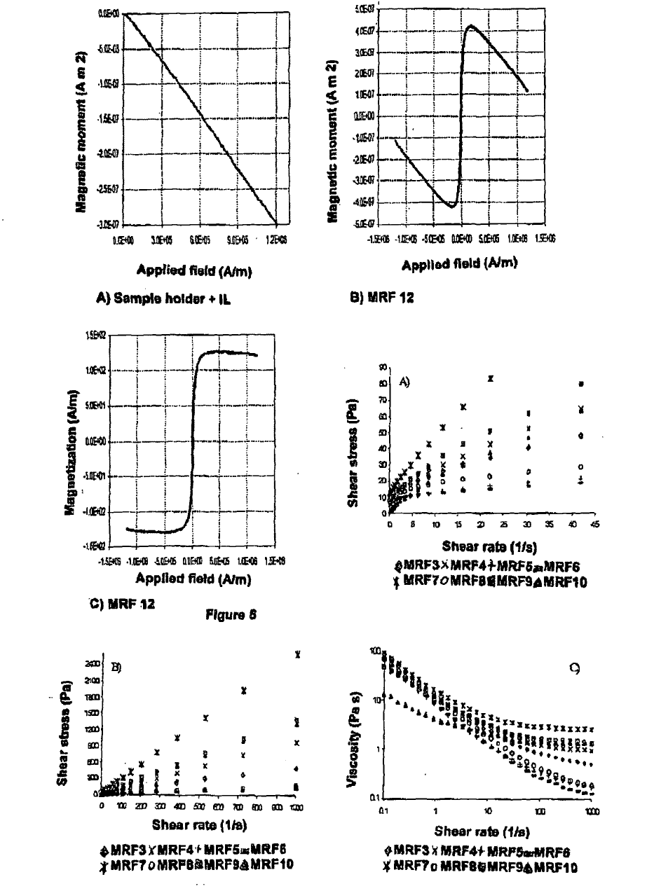

FIG. 3 shows a representative magnetic hysteresis loop (MRF3) of the obtained magnetic measurements of the investigated MRFs. In order to investigate whether the utilized ILs as carriers have any influence on the magnetic properties of the prepared MRFs, magnetization measurements of the investigated ILs (pure) as well as the sample holder (glass) were performed.

FIG. 4 shows the results of these measurements for the case of IL4 which clearly reveals the diamagnetic characteristics (low magnetic moment values even at high magnetic fields) for both, the sample holder and IL4.

According to the results shown in FIG. 4, one can conclude that the contribution of the ILs to the magnetic properties of the prepared MRFs is negligible.

It was shown above that for MRFs with a relatively high content of magnetic particles the diamagnetic properties shown by the sample holder as well as the used ILs have a negligible influence on the magnetic properties of the MRF. However, for fluids in which the content of dispersed magnetic material is very low, even the weak magnetic properties shown by the sample holders and the carriers (both diamagnetic materials) can have a considerable influence on the determination of the properties of the sample. This influence was investigated with sample MRF12, which content of magnetic particles is 0.2% wt., and the obtained results are illustrated in FIG. 5.

The magnetization measurements for a sample holder containing IL8 (pure) and the sample MRF12 are shown in FIGS. 5A and 5B, respectively. It is obvious that at this low content of dispersed magnetic material both, the sample holder and the carrier, have an influence on the magnetization measurements and therefore the magnetization properties cannot be determined directly from the magnetic hysteresis loop shown in FIG. 5B. Owing to this influence, a correction on the original magnetic hysteresis loop (FIG. 5B) must be performed. This can be achieved by subtracting the magnetic moments of the sample holder containing the carrier (FIG. 5A) from the magnetic hysteresis loop of FIG. 5B; the result of this correction is shown in FIG. 5C (including the division of the magnetic moments by the volume of the analyzed sample). Finally, from FIG. 5C the magnetic properties of sample MRF12 were determined and the results are reported in Table 2.

As already mentioned, the main characteristic of MRFs is the reversible modification of their rheological properties by means of a magnetic field. With regard to this property, magnetorheological measurements were performed for some of the prepared MRFs. For these measurements only MRFs with a content equal or greater then 8.5% wt. of micron-sized magnetic particles were analyzed and the obtained results are discussed below. The discussion is mainly based on the results obtained a constant temperature (25° C.). In general, all the analyzed samples showed a reversible increase on their viscosity and shear stress (up to 2 orders of magnitude in some cases) when were subjected to a magnetic field.

Basically, there are two contributions to the rheology of the prepared MRFs based on ILs: the contribution related to rheological properties of the pure ILs as carriers and the contribution related to the dispersed magnetic particles in the fluid. On the one hand, it is known that most of the ILs are more viscous than common solvents, that small amounts of impurities can have important effect on their viscosity, and that, in general, most ILs show Newtonian behavior. On the other hand, it is reported in literature that the viscosity of suspensions will vary from that of the carrier liquid due to the presence of the suspended particles. Thus, the viscosity of the prepared MFRs in absence of a magnetic field is then determined by the viscosity of the carrier IL and the volume fraction of suspended magnetic material (φ) according to the established theories of viscosity of suspensions.

Rheological measurements in absence of a magnetic field and at 25° C. revealed that the analyzed MRFs based on ionic liquids show a “quasi-Newtonian” behavior. In other words, the dispersions show a slight pseudo-plastic behavior (shear thinning) at low shear rates as shown in FIG. 6A. However, the dispersions become

Newtonian-like (linear dependences in shear stress vs. shear rate plots) for shear rates greater than 16 s⁻¹ as illustrated in FIG. 6B. FIG. 6C shows a logarithmic plot of the viscosity vs. shear rate for analyzed MRFs at the mentioned experimental conditions. The influence of the concentration of the dispersed magnetic particles on the measured rheological properties can also be seen in FIG. 6 for the samples MRF9 and MRF10 (25 and 8.5% wt of particles, respectively, both in IL8). As expected, FIG. 6 reveal lower viscosity values for the less concentrated dispersion (MRF10) especially at low shear rates.

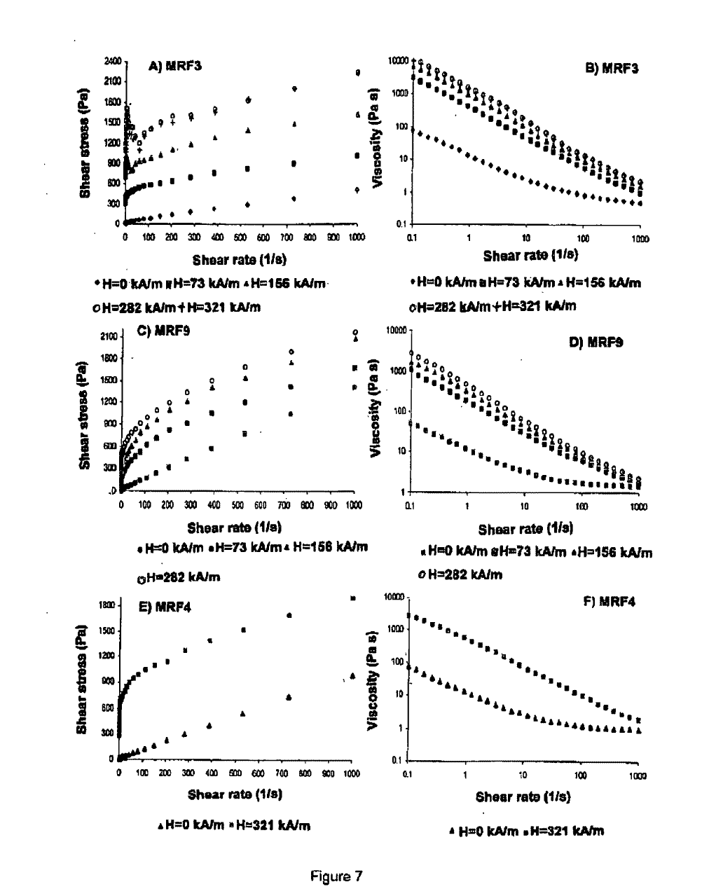

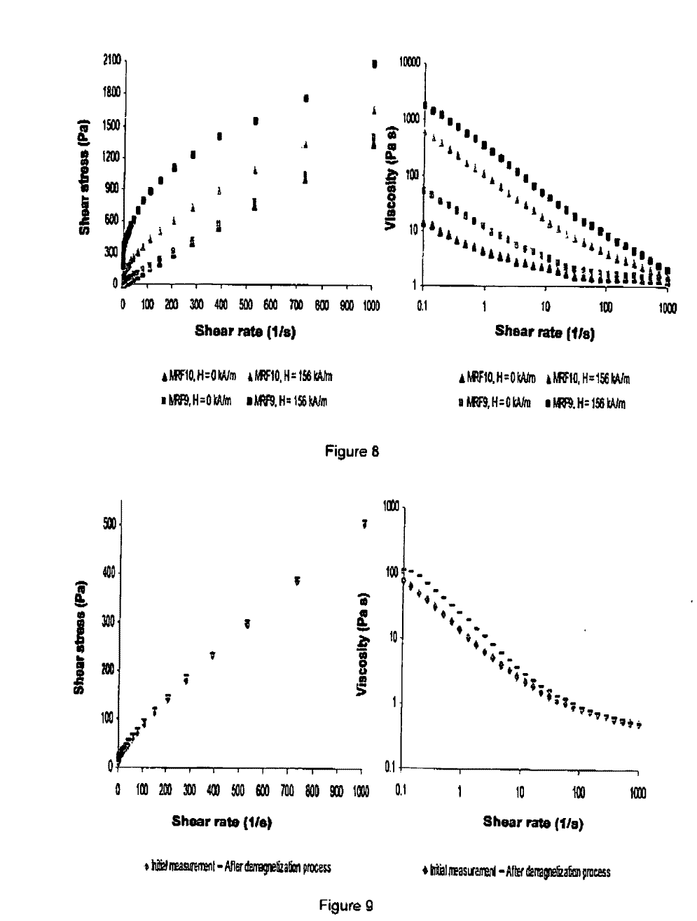

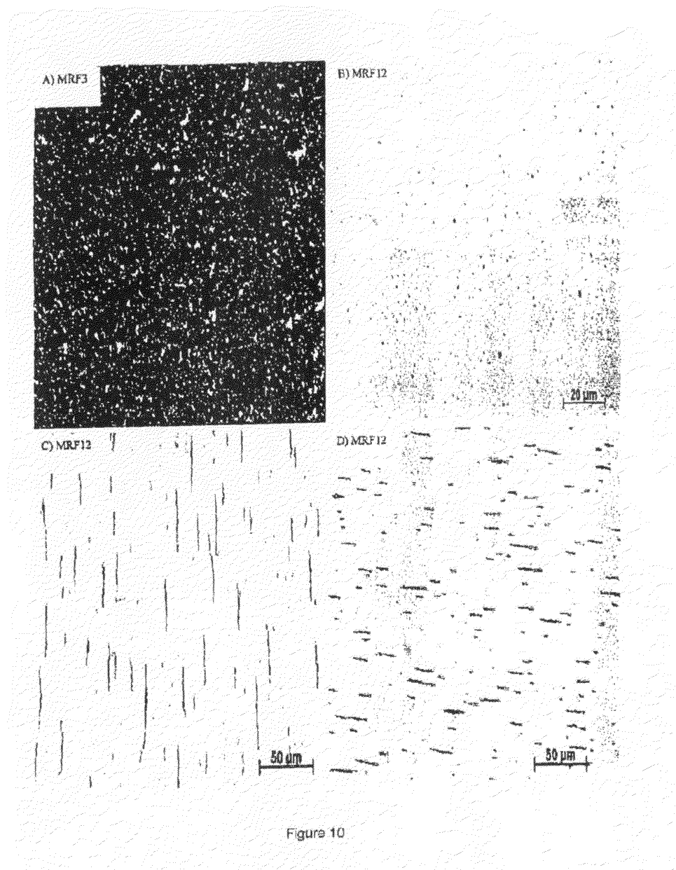

FIG. 7 reveals that the rheological properties of the prepared MRFs can be modified by the means of a magnetic field. Thus, the shear stresses and the viscosities values of the investigated MRFs increase as the intensity of the applied magnetic field increases up to intensities of magnetic fields where the saturation magnetizations of the samples are reached (FIG. 3). For example, in FIGS. 7A and 7B it can be seen that the maximum shear stresses and viscosities values are reached with a magnetic field of 282 kA/m and the use of a higher intensities (321 kA/m) does not have a considerable effect on the rheological properties any more since the saturation magnetization of the sample was already reached. From FIG. 7 it is noted that the investigated MRFs show a plastic or Bingham type behavior in the presence of a magnetic field and the observed yield stresses (=minimum shear stresses below which no shear flow takes place) depend on the strength of the applied magnetic field as reported in the literature. In general, this property of MRFs enables the design of technical applications and engineering devices (e.g. dampers). FIG. 7 also shows that depending on the IL used during the preparation of the magnetic dispersions, the corresponding MRF can also show a highly non-linear behavior in the shear stress vs. shear plots (FIG. 7A, MRF3 at low shear rates and intermediate intensities of magnetic field) apart from described typical Bingham behavior (FIGS. 7C and 7E). The viscosity data shown in FIG. 7 (FIGS. 7B, 7D and 7F) for measurements performed in the presence of a magnetic field follow in good agreement the power law model, which is the simplest and frequently used law for describing non-Newtonian fluids.

The influence of the content of the dispersed magnetic particles in the investigated MRFs on the rheological properties in the absence and in the presence of a magnetic field is depicted in FIG. 8. As expected, the less content of magnetic material in a same carrier (IL 8) the lower the values of viscosity and shear stress for a fixed strength of magnetic field.

Finally, FIG. 9 confirms that the modification of rheological properties of the analyzed MRFs is a reversible process and that any rheological change induced in the fluids due to the presence of a magnetic field must vanish in absence of this one.

FIG. 9 shows an initial measurement performed before the application of any magnetic field to the sample (MRF3 in this case) and another one of the same sample after its analysis at different strengths of magnetic fields and after the application of a demagnetization process in the used rheometer. Both measurements in FIG. 9 reveal only slightly differences from each other confirming the reversibility of the process. This slightly differences can be attributed to the remanent magnetizations shown by the samples as illustrated in FIG. 3, which must vanish completely whether the time between measurements is long enough. The rest of the analyzed MRFs showed similar behavior to that one depicted in FIG. 9.

FIG. 10 shows optical microscopy images for two of the prepared MRFs, for the case of one of the highest concentration of dispersed magnetic particles investigated and for the case of the lowest concentration. FIG. 10A displays an image of the sample MRF3 (25% wt of dispersed magnetic particles in IL 2), which reveals that the particles are well dispersed and form a homogeneous and stable mixture in the absence of a magnetic field. FIGS. 10B, 10C, and 10D show images of the sample MRF12 (0.2% wt of dispersed magnetic particles in IL 8); in these cases the low concentration of particles in the sample allows for a better analysis of the dispersions. On the one hand, in FIG. 10B can be observed that in the absence of a magnetic field the inter-particle interactions are negligible and the used particles are around 1 μm in diameter. On the other hand, if a magnetic field is applied to the sample inter-particle interactions becomes significant that complex structures and large chains or rods of magnetic particles are formed (FIGS. 10C and 10D), which will determine the rheological behavior of the MRFs. The mentioned structures are aligned parallel to the direction of the applied magnetic field.

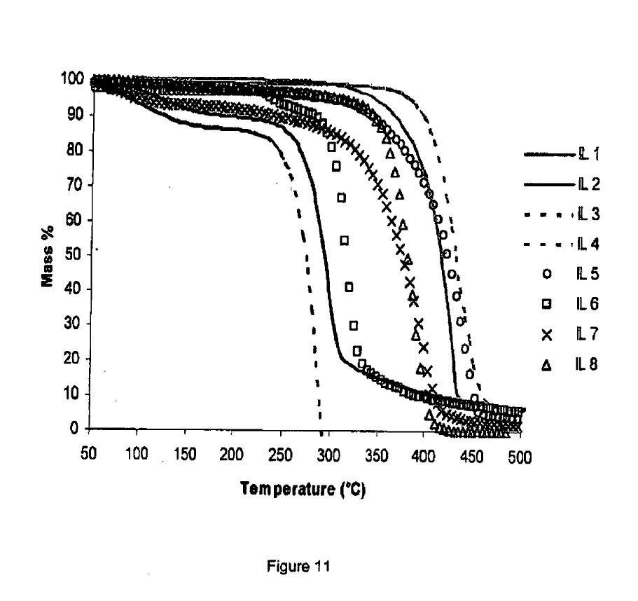

Thermogravimetric measurements of the utilized ILs for the preparation of the MRFs of this work reveal that the magnetic fluids of this invention expands the applications of magnetorheological technology in high temperature processes since most of the investigated ILs show a good thermal stability up to 250° C. and in some cases nearly up to 400° C., as shown in FIG. 11 for the case of IL 4.

1-24. (canceled)

25. A magnetic fluid comprising:
a. a liquid chosen from the group consisting of ionic liquids, mixtures of ionic liquids, stabilized ionic liquids, and stabilized mixtures of ionic liquids;
b. magnetic particles dispersed in said liquid; and
wherein said stabilized ionic liquids and said stabilized mixtures of ionic liquids, if present, further comprise a stabilization agent selected from the group consisting of carbon fibers, natural and synthetic water-soluble thixotropic agents, resins, starches, polysaccharides, cellulose derivatives, sodium tetraborate decahydrate, seaweed extracts, synthetic resins, surfactants, viscoplastic media, water-in-oil emulsions and combinations thereof.

26. A magnetic fluid according to claim 25, wherein the magnetic particles are present in the fluid in an amount of between 1 and 60% of the total weight of the magnetic fluid.

27. A magnetic fluid according to claim 25, wherein the magnetic particles have diameters in the range between 10 nm and 50 μm.

28. A magnetic fluid according to claim 25, wherein the magnetic particles have diameters in the range between 100 nm and 20 μm.

29. A fluid according to claim 25, wherein the magnetic particles are selected from the group consisting of particles showing ferromagnetic, ferrimagnetic, antiferromagnetic, canted-spin ferromagnetic, paramagnetic or superparamagnetic properties and mixtures thereof.

30. A magnetic fluid according to claim 25, wherein the magnetic particles are present in the fluid in an amount of between 5 and 40% of the total weight of the magnetic fluid.

31. A magnetic fluid according to claim 25 wherein at least one stabilization agent is present in an amount, up to 40% by weight of the total amount of the liquid, which amount is effective to render said magnetic fluid soft settling.

32. A magnetic fluid according claim 31, wherein said stabilization agent comprises at least one polymer.

33. A fluid according to claim 25, wherein the liquid consists of at least 95% by weight of ions and has a melting point between −100 and +200° C.

34. A fluid according to claim 33, wherein the liquid comprises cations selected from the group consisting of: an ammonium cation with at least one organic group: a phosphonium cation with at least one organic group or a sulfonium cation with at least one organic group: a quaternary nitrogen atom containing a heterocyclic group and mixtures of two or more thereof.

35. A fluid according to claim 33, wherein the liquid comprises cations selected from the group consisting: of N-alkylpyridinium, N-alkyl-vinylpyridinium, tetraalkylammonium, tetraalkylphosphonium, trialkyl-sulfonium, N,N′-dialkylimidazolium, N-alkyl-N′-vinylimidazolium and mixtures of two or more thereof.

36. A fluid according to claim 33, wherein the liquid comprises anions selected from the group consisting of an adduct of a Lewis acid, a trihalide, and a halide.

37. A fluid according to claim 33, wherein the liquid comprises anions selected from the group consisting of fluoride, chloride, bromide, iodide, hexafluorophosphate, hexafluoroantimonate, hexafluoroarsenate, tetrafluoroborate, dicyandiamide, methanesulfonate, tosylate tetrachloroborates, tetraarylborates, polyfluorinated tetraarylborates, tetrahaloaluminates, alkyltrihaloaluminates, triflate, nonaflate, bistrifylimides, chloroacetate, trifluoroacetate, alkylsulfates, N—(N-methoxyethoxy)alkylsulfates, dialkylphosphates, [MeCO₂]”, nitrate, nitrite, trichlorozincate, dichlorocuprate, fluorosulfonate, triarylphosphine, sulfonates, polyhedral boranes, carboranes, metallocarboranes and mixtures thereof.

38. A magnetic fluid according to claim 33, wherein at least one stabilization agent is present in an amount, up to 40% by weight of the total amount of the liquid, which amount is effective to render said magnetic fluid soft settling.

39. A magnetic fluid according to claim 38, wherein said stabilization agent comprises at least one polymer.

40. A magnetic fluid according to claim 33, wherein the magnetic particles are present in the fluid in an amount of between 1 and 60% of the total weight of the magnetic fluid.

41. A fluid according to claim 40, wherein the magnetic particles are selected from the group consisting of iron, carbonyl iron, iron alloys, iron oxides, iron nitrides, iron carbides, low-carbon steel, nickel, cobalt, rare-earths, mixtures and alloys thereof.

42. A fluid according to claim 41, wherein the magnetic particles are materials selected from the group consisting of iron, iron oxides, cobalt, nickel, gadolinium and their ferromagnetic and ferrimagnetic alloys.

43. A magnetic fluid according to claim 42, wherein the magnetic particles are present in the fluid in an amount of between 1 and 60% of the total weight of the magnetic fluid.

44. A magnetic fluid according to claim 43, wherein at least one stabilization agent is present in an amount, up to 40% by weight of the total amount of the liquid, which amount is effective to render said magnetic fluid soft settling.

45. A magnetic fluid according to claim 44, wherein said stabilization agent comprises at least one polymer.

46. A magnetic fluid according to claim 42, wherein the magnetic particles are present in the fluid in an amount of between 5 and 40% of the total weight of the magnetic fluid.

47. An magnetic fluid ink, suitable for ink jet printing, comprising:
c. a liquid chosen from the group consisting of ionic liquids, mixtures of ionic liquids, stabilized ionic liquids, and stabilized mixtures of ionic liquids;
d. magnetic particles dispersed in said liquid;
wherein said stabilized ionic liquids and said stabilized mixtures of ionic liquids, if present, further comprise a stabilization agent selected from the group consisting of carbon fibers, natural and synthetic water-soluble thixotropic agents, resins, starches, polysaccharides, cellulose derivatives, sodium tetraborate decahydrate, seaweed extracts, synthetic resins, surfactants, viscoplastic media, water-in-oil emulsions and combinations thereof.

48. An improved electromagnetic device using a damping fluid, said device being chosen from the group consisting of loudspeakers, graphic plotters and instrument gauges, wherein the improvement comprises provision of a magnetic fluid damping fluid, said magnetic fluid damping fluid comprising:
e. a liquid chosen from the group consisting of ionic liquids, mixtures of ionic liquids, stabilized ionic liquids, and stabilized mixtures of ionic liquids;
f. magnetic particles dispersed in said liquid;
wherein said stabilized ionic liquids and said stabilized mixtures of ionic liquids, if present, further comprise a stabilization agent selected from the group consisting of carbon fibers, natural and synthetic water-soluble thixotropic agents, resins, starches, polysaccharides, cellulose derivatives, sodium tetraborate decahydrate, seaweed extracts, synthetic resins, surfactants, viscoplastic media, water-in-oil emulsions and combinations thereof.

49. An improved electromagnetic device, chosen from the group consisting of gas lasers, motors, blowers and hard drives, comprising a sealing fluid, wherein the improvement comprises provision of a magnetic fluid sealing fluid, said magnetic fluid sealing fluid comprising:
g. a liquid chosen from the group consisting of ionic liquids, mixtures of ionic liquids, stabilized ionic liquids, and stabilized mixtures of ionic liquids;
h. magnetic particles dispersed in said liquid;
wherein said stabilized ionic liquids and said stabilized mixtures of ionic liquids, if present, further comprise a stabilization agent selected from the group consisting of carbon fibers, natural and synthetic water-soluble thixotropic agents, resins, starches, polysaccharides, cellulose derivatives, sodium tetraborate decahydrate, seaweed extracts, synthetic resins, surfactants, viscoplastic media, water-in-oil emulsions and combinations thereof.

50. An improved process using a fluid, said process being chosen from the group consisting of:
i. performing chemical reactions;
j. transporting and delivering chemical substances to a selected location within a chemical or biological system;
k. occluding blood vessels in living organisms in medical therapy;
l. conducting chemical reactions wherein the diffusion of the involved reactants is controlled by controlling the viscosity of the reaction media;
m. biomedical applications, chosen from the group consisting of drug targeting, cell labeling and attaching drugs to magnetic particles;
n. sink flotation recovery of resources from waste materials; and
o. imaging processes,
wherein the improvement comprises use of magnetic fluid therein, wherein the magnetic fluid comprises:
i. a liquid chosen from the group consisting of ionic liquids, mixtures of ionic liquids, stabilized ionic liquids, and stabilized mixtures of ionic liquids;
ii. magnetic particles dispersed in said liquid; and
wherein said stabilized ionic liquids and said stabilized mixtures of ionic liquids, if present, further comprise a stabilization agent selected from the group consisting of carbon fibers, natural and synthetic water-soluble thixotropic agents, resins, starches, polysaccharides, cellulose derivatives, sodium tetraborate decahydrate, seaweed extracts, synthetic resins, surfactants, viscoplastic media, water-in-oil emulsions and combinations thereof.
